(19) United States
(12) Patent Application Publication
(10) Pub. No.: US 2026/0319196 A1
(43) Pub. Date: Oct. 15, 2026

(54) OPTICAL FIBER CONNECTORS AND TOOLS FOR ASSEMBLY THEREOF

(71) Applicant: Alcon Inc., Fribourg (CH)

(72) Inventors: Neda Dadashzadeh, MIssion Viejo, CA (US); Nathan Ty Nguyen, Anaheim, CA (US); Qing Xiang, Irvine, CA (US); Adela Apostol, Laguna Hills, CA (US)

(21) Appl. No.: 19/461,884

(22) Filed: Jan. 28, 2026

Related U.S. Application Data
(60) Provisional application No. 63/763,371, filed on Feb. 26, 2025.

(51) Int. Cl. G02B 6/42; A61F 9/007

(52) U.S. Cl. CPC G02B 6/424; A61F 9/007

(57) ABSTRACT

Embodiments disclosed herein provide a connector for an ophthalmic surgical device. The connector includes a housing, an optical fiber, a ferrule body, a protective tube, and a ferrule. The housing is configured to be gripped by a user for coupling to a surgical console. The optical fiber is disposed within the housing, the ferrule body is disposed around at least a portion of the optical fiber, and the protective tube is disposed within the ferrule body. The ferrule is coupled to the ferrule body and the protective tube. The ferrule defines a cavity at a proximal end of the ferrule, and the optical fiber is at least partially disposed within the cavity.

### INTRODUCTION

Microsurgical procedures frequently involve the use of illumination light and/or laser light (e.g., laser treatment beam (“treatment beam”), laser aiming beam (“aiming beam”), etc.) during the treatment of various body tissues. For example, certain ophthalmic surgical procedures may involve using illumination light to visualize tissues within the eye and/or using laser light to cauterize or emulsify the tissues within the eye. During such procedures, an illumination probe and/or a laser probe may be used to help the surgeon treat and/or illuminate an area of treatment within the eye.

Typically, one or more optical fibers housed within an optical cable are used to transmit light that is propagated by a surgical system to the illumination or laser probe. In such cases, a connector is usually used to couple the proximal end of the optical cable to a port of the surgical laser system (e.g., housed by a surgical console), while at its distal end, the optical cable couples to the probe. However, poor alignment between the port and the connector can result in a loss of optical power and efficiency associated with the laser or illumination light propagated by the surgical system. Consequently, the probe may not provide adequate laser and/or illumination light within the eye, thereby restricting the surgeon’s ability to treat and visualize the treatment area.

### BRIEF SUMMARY

The present disclosure relates generally to connectors for optical fibers and tools for assembly thereof.

In certain embodiments, a connector for an ophthalmic surgical device is provided. The connector includes a housing, an optical fiber, a ferrule body, a protective tube, and a ferrule. The housing is configured to be gripped by a user for coupling to a surgical console. The optical fiber is disposed within the housing, the ferrule body is disposed around at least a portion of the optical fiber, and the protective tube is disposed within the ferrule body. The ferrule is coupled to the ferrule body and the protective tube. The ferrule defines a cavity at a proximal end of the ferrule, and the optical fiber is at least partially disposed within the cavity.

In certain embodiments, connector assembly for an ophthalmic surgical device is provided. The connector assembly includes a cable configured to be coupled with the ophthalmic surgical device, and a connector coupled to a proximal end of the cable. The connector includes a housing, an optical fiber, a ferrule body, a protective tube, and a ferrule. The housing is configured to be gripped by a user for coupling to a surgical console. The optical fiber is disposed within the housing, the ferrule body is disposed around at least a portion of the optical fiber, and the protective tube is disposed within the ferrule body. The ferrule is coupled to the ferrule body and the protective tube. The ferrule defines a cavity at a proximal end of the ferrule, and the optical fiber is at least partially disposed within the cavity.

In certain embodiments, a connector assembly tool is provided. The connector assembly tool includes a base and a semi-circular mount protruding laterally from the base. The base includes a first semi-circular portion configured to hold an optical fiber. The semi-circular mount includes a second semi-circular portion configured to hold a protective tube, and a third semi-circular portion configured to hold a ferrule.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A shows an example system, including an example surgical laser system for generating laser beams for delivery to a surgical target, according to certain embodiments.

FIG. 1B shows example components of the surgical laser system of FIG. 1A, according to certain embodiments.

FIGS. 2A-2B show cross-sectional side views of an example port and an example connector assembly in an unmated state and a mated state, respectively, according to certain embodiments.

FIG. 3A shows a perspective view of an example connector assembly, according to certain embodiments.

FIG. 3B shows a cross-sectional side view of the connector assembly of FIG. 3A, according to certain embodiments.

FIG. 3C shows a cross-sectional side view of example components of the connector assembly of FIG. 3B, according to certain embodiments.

FIG. 4 shows a perspective view of an example connector assembly tool, according to certain embodiments.

FIG. 5 shows a perspective view of an example ferrule body holder tool, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

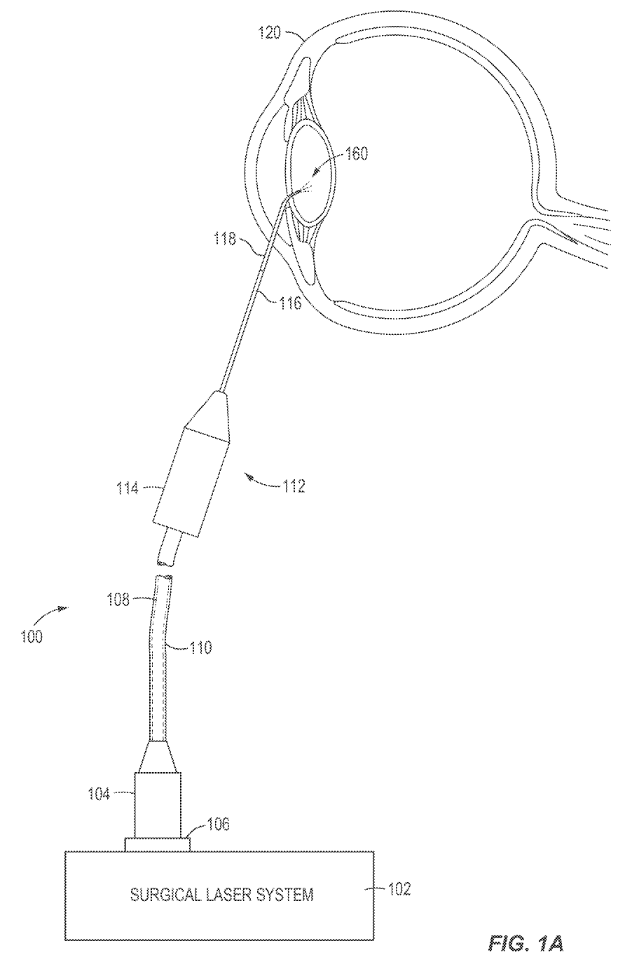

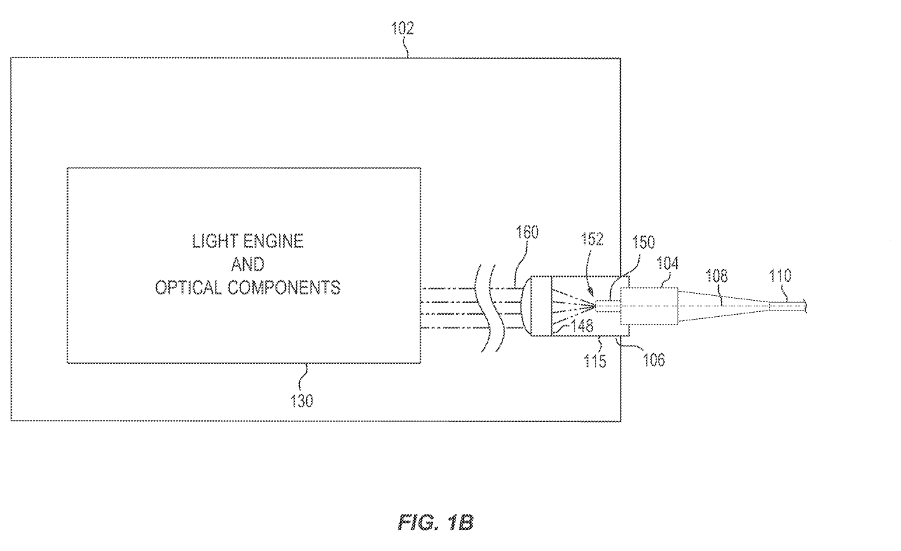

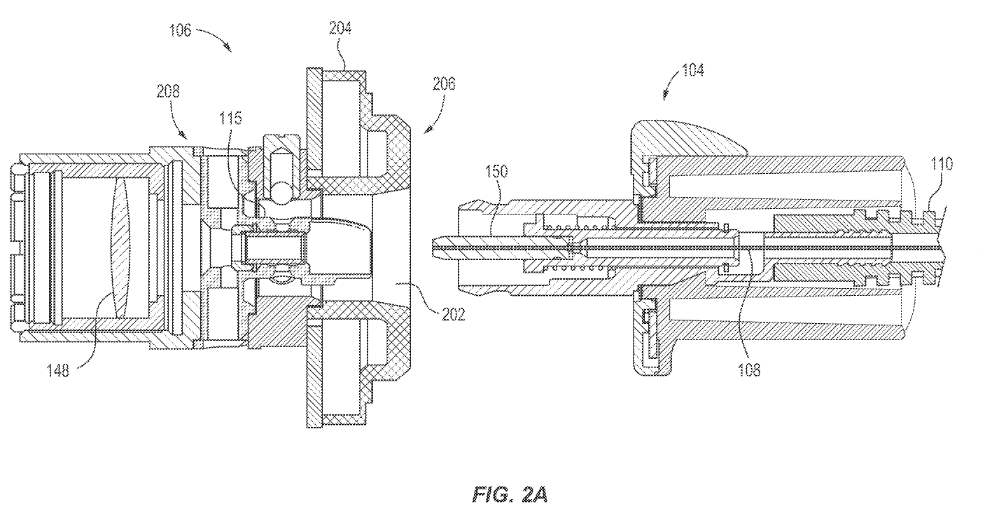

As discussed above, during ophthalmic surgeries, illumination light (e.g., illumination beam) and/or laser light (e.g., treatment laser beam, aiming laser beam, etc.) are often used to treat and/or illuminate a treatment area within an eye. As an example, an illumination probe is often used to illuminate an interior portion of the eye for treatment by the surgeon. As another example, a laser probe is used to cut, cauterize, and/or emulsify tissues within the eye. In each example, light (e.g., illumination light or laser light) may be generated by a light and/or laser source in a surgical laser system (or a surgical console), and transmitted through an optical fiber to the probe (e.g., illumination probe or laser probe). At its proximal end, the optical fiber couples to a connector that connects the optical fiber to a port of the surgical laser system, and at its distal end the optical fiber couples to the probe, which is manipulated by a surgeon. A system and various components thereof are described herein with reference to FIGS. 1A-1B and 2A-2B.

FIG. 1A shows an example system 100 for performing a laser-assisted ophthalmic procedure, according to certain embodiments. The system 100 includes a surgical laser system 102 having one or more laser sources for generating laser beams for transmission by a laser probe 112. As an example, the surgical laser system 102 may be integrated in a surgical console for performing ophthalmic surgical procedures (e.g., cataract surgeries, corneal transplants, glaucoma surgeries, LASIK (Laser-Assisted In Situ Keratomileusis) surgeries, refractive lens exchanges, vitreoretinal procedures, trabeculectomies, keratotomy procedures, and/or keratoplasty surgeries, etc.).

As shown in FIG. 1A, the probe 112 includes a probe body 114, a probe tip 116, and a distal end portion 118. The probe 112 is coupled to the surgical laser system 102 via a connector 104 and a cable 110. The connector 104 is coupled to a port 106 of the surgical laser system 102. The cable 110 includes an optical fiber 108 therein having a distal end that couples to and extends through the probe 112 and a proximal end that couples to and extends through the connector 104. The optical fiber 108 delivers at least one beam 160 generated by the surgical laser system 102 to the probe 112, which transmits the beam 160 to a patient’s eye 120.

FIG. 1B shows example components included in the surgical laser system 102, according to certain embodiments. The surgical laser system 102 comprises a light engine (e.g., one or more laser sources) and optical components 130 that propagate the beam 160. As an example, the beam 160 includes a treatment beam for treating tissue and/or an aiming beam for illuminating targeted treatment areas. In some embodiments, the light engine also includes an illumination source for propagating, with the beam 160, an illumination beam for illuminating the inside of the eye.

The optical components 130 of the surgical laser system 102 may include a plurality of lenses, diffractive elements, beam splitters, and other optical relay devices for polarizing, isolating, collimating, selecting, dividing, combining, relaying, and condensing the laser and/or illumination beams between their respective sources and desired ports, which may together be referred to as an “optical relay system.”

The beam 160 is propagated and directed by the light engine and optical components 130 to a condensing lens 148. The condensing lens 148 is a multi-element achromat comprising two singlet lenses and one doublet lens. The condensing lens 148 converges the beam 160 into the optical fiber 108, which is coupled to a port adapter 115 at the port 106 of the surgical laser system 102 via the connector 104. Together, the port 106 and the condensing lens 148 may be referred to as a “chimney.”

The port adapter 115 is disposed adjacent to the condensing lens 148 and is configured to mate with the connector 104 to couple the optical fiber 108 to the surgical laser system 102. The condensing lens 148 focuses the beam 160 into the optical fiber 108 such that the beam 160 is propagated to the distal end of a surgical probe (e.g., probe 112) that is coupled to the optical fiber 108. As described above, the proximal end of the optical fiber 108 is exposed through an opening 152 of the port adapter 115, respectively, via a ferrule 150 of the connector 104.

FIGS. 2A-2B illustrate cross-sectional side views of the port 106 and the connector 104 of FIGS. 1A-1B in an unmated state and a mated state, respectively, according to certain embodiments.

As shown, the port 106 includes the port adapter 115, which is at least partially disposed through an opening 202 in a cover plate 204 at a distal end 206 of the port 106. In certain embodiments, the cover plate 204 couples to an outer panel of a surgical laser system (e.g., the surgical laser system 102). An opposing, proximal end 208 of the port 106 is disposed adjacent to the condensing lens 148, which is configured to focus the beam 160 onto the optical fiber 108 disposed within connector 104 as described with reference to FIG. 1B.

The connector 104 is a push-pull connector that includes the ferrule 150 and the optical fiber 108 disposed within the connector 104. The ferrule 150 and the optical fiber 108 are angularly aligned and angularly fixed within the connector 104. From the ferrule 150, the optical fiber 108 extends distally through the connector 104 and into the cable 110, which protects and couples optical fiber 108 with a surgical probe (e.g., probe 112).

As shown in FIG. 2B, when fully inserted into the port 106, the ferrule 150 is disposed within the port adapter 115 such that the condensing lens 148 converges the beam 160 into the optical fiber 108. During insertion, the port adapter 115 and the connector 104 mechanically guide the lateral position and rotational orientation of the connector 104 and the ferrule 150. When fully inserted into the port 106, the connector 104 facilitates alignment of the optical fiber 108 with the condensing lens 148, such that the beam 160 converges into the optical fiber 108.

In the example of FIGS. 2A-2B, the connector 104 is a push-pull connector, which allows for easier insertion and coupling with the port 106, for example, relative to SubMiniature version A (SMA) connectors and other types of optical fiber connectors. For example, although SMA connectors can be used for high-power laser applications, these connectors typically require a skilled assistant to ensure proper alignment of the connector with the port. If the SMA connector is not properly aligned with the port, it can result in the loss of transmission efficiency (i.e., loss of some light) as light propagates out of the port and into the connecter. As such, push-pull connectors, such as connector 104, are used as shown in FIGS. 2A-2B to improve proper alignment between the connector and the port.

However, current push-pull connectors can typically only be used with certain optical fibers, such as those used in low-power laser applications (e.g., involving power outputs less than or equal to 1.5 watts (W)), because other optical fibers, such as those used in high-power laser applications (e.g., involving power outputs greater than 1.5 W), are fragile and often break during assembly of the push-pull connectors, or while the push-pull connector is coupled to the port.

For example, the optical fiber 108 may break in response to external forces moving the connector 104 and/or the cable 110 while coupled to the port 106. As an example, if the push-pull connector 104 and/or the cable 110 are moved in any direction other than into/out of the port 106, then the optical fiber 108 typically breaks because it is held in position by the ferrule 150 and the port adapter 115. In other words, the optical fiber 108 breaks because it is locked in position by the ferrule 150, which is disposed in the port adapter 115, and is unable to bend with the rest of the connector 104 and/or cable 110 when moved.

Similarly, during the assembly of the connector 104, the optical fiber 108 often breaks during insertion into the ferrule 150, or due to bending of the optical fiber 108 while other components of the push-pull connector are put together. As such, the optical fiber 108 is then replaced with a new optical fiber. However, when the optical fibers are replaced, they typically demonstrate inconsistent alignment (e.g., relative to one another) because each optical fiber couples with the ferrule 150 differently. Consequently, the connector 104 may not provide the same optical power and efficiency after its optical fiber is replaced.

Further, current push-pull connectors can typically only be used in low-power laser applications, for example, that involve power outputs less than or equal to 1.5 W, because assembly of the push-pull connector 104 involves the use of an adhesive at a proximal end of the optical fiber 108 disposed within the ferrule 150. In other words, current push-pull connectors are limited to low-power laser applications because high-power laser applications typically involve power outputs greater than 1.5 W, which melts the adhesive at the proximal end of the optical fiber 108, thereby subjecting the optical fiber 108 to move, and possibly dislocate from the ferrule 150.

Accordingly, the embodiments described herein provide an improved connector and tools for the assembly thereof. For example, the connector described herein structurally supports a wide range of optical fibers and circumvents the need for adhesives at the proximal ends of the optical fibers. The connector can also be used in low-power and high-power laser applications, and facilitates proper alignment with the port without the need for a skilled assistant. Further, the tools for assembling the connector herein preserve alignment of the optical fiber therein, prevent the optical fiber from breaking, and maintain consistent alignment when the optical fiber is replaced, thereby maintaining constant optical power and efficiency across each assembled connector.

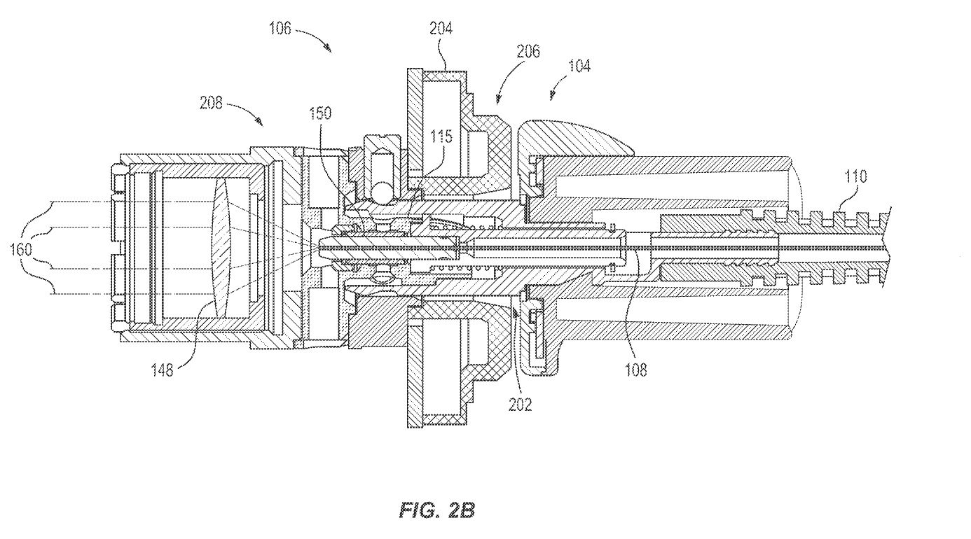

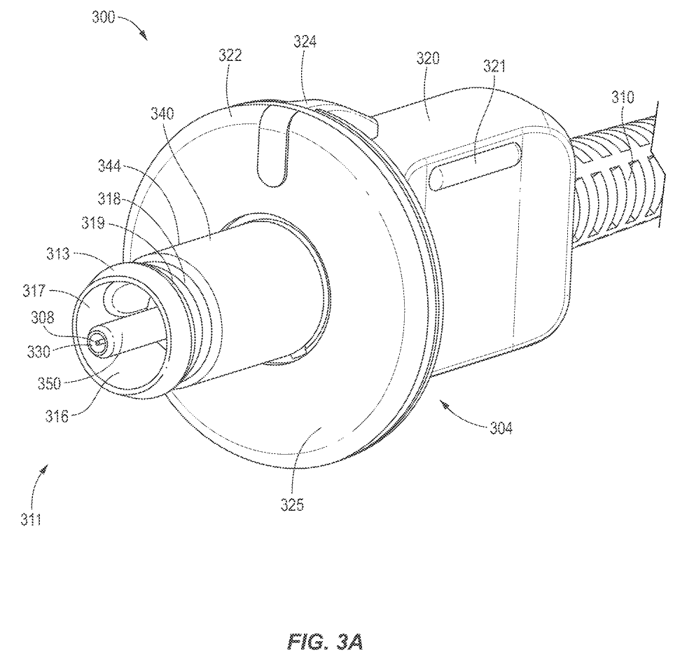

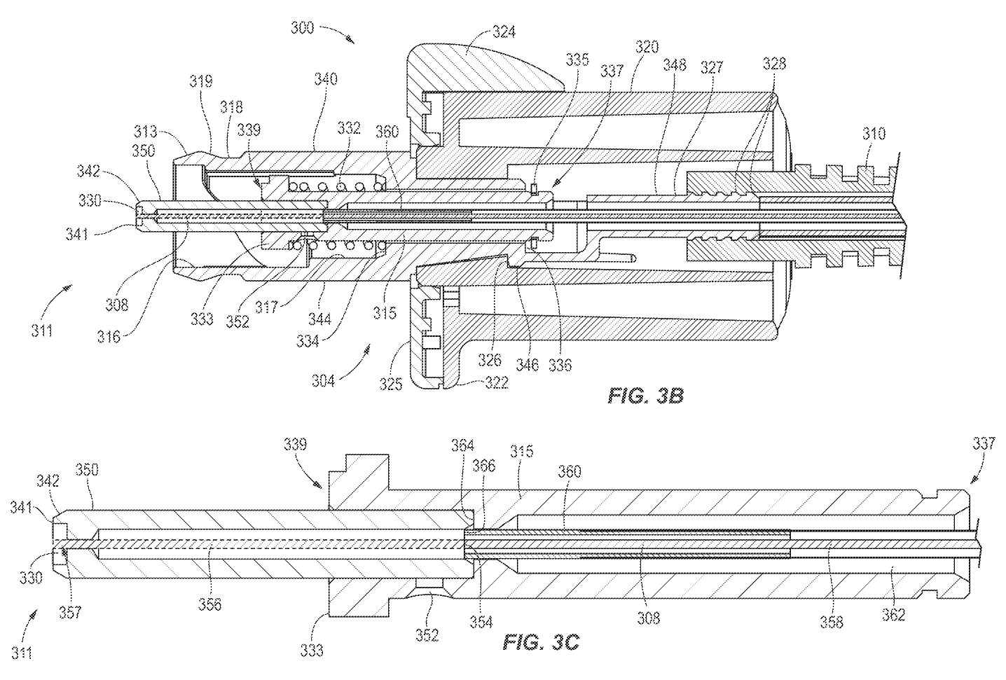

FIG. 3A shows a perspective view of an example connector assembly 300, according to certain embodiments. FIG. 3B shows a cross-sectional side view of the connector assembly 300 of FIG. 3A, according to certain embodiments. FIG. 3C shows a cross-sectional side view of example components of the connector assembly 300 of FIG. 3A, according to certain embodiments. Accordingly, FIGS. 3A-3C are described together herein for clarity purposes.

As shown in FIGS. 3A-3B, the connector assembly 300 includes a connector 304 and a cable 310 coupled to the connector 304, the connector 304 and the cable 310 comprising portions of an optical fiber 308. The connector 304, similar to the connector 104, is a push-pull connector configured to releasably mate with a port (e.g., port 106) via a push-pull retention mechanism to couple the optical fiber 308 to a light engine of a surgical laser system (e.g., surgical laser system 102). As such, the connector 304 typically does not require a skilled assistant to couple the connector 304 to the port. For example, as opposed to SMA connectors, the connector 304 achieves proper alignment upon insertion into the port and thereby maintains transmission efficiency as light propagates into the connector 304 from the port.

The connector 304 includes a block-like housing 320 having an outer surface ergonomically contoured and configured to be gripped by a user, such as a surgeon or surgical assistant, for coupling the connector 304 to a surgical laser system (e.g., surgical laser system 102). The outer surface of the housing 320 may be textured or have one or more gripping features formed thereon, such as one or more grooves and/or ridges 321. The housing 320 couples to a proximal sterility flange 322, which protects against contact of a user’s surgical gloves with, e.g., a non-sterile surgical system and/or surgical console. A cap 325 is snap-fit to the sterility flange 322 and/or the housing 320, for example, via one or more snap-fit connectors configured to interlock with each other for efficient assembly of the two components.

The connector 304 further include(s) a prominent fin-shaped rib 324 that extends distally from the cap 325, through a slot in the sterility flange 322, and along an outer surface of the housing 320. The fin-shaped rib 324 may be molded in a contrasting color relative to a color of the housing 320, the sterility flange 322, and/or the cap 325 for enhanced clarity for the user. The fin-shaped rib 324 also assists a user in orienting the connector 304 for initial insertion into the port.

The connector 304 further includes a barrel member 340, which extends through the housing 320 and proximally outward therefrom. The barrel member 340 includes an external and tubular proximal connector portion 344 configured to be inserted into the port, a medial portion 346 for fixedly coupling the barrel member 340 to the housing 320, and a distal connector portion 348 for coupling the connector 304 to the cable 310. As shown in FIG. 3B, the medial portion 346 is secured to the housing 320 via one or more snap-fit connections 326 or other retaining mechanisms. The distal connector portion 348 includes one or more features 328 (e.g., one or more ridges, threads, or ribs) formed on an outer surface 327 of the distal connector portion 348 for securing the cable 310 thereon.

The proximal connector portion 344 helps facilitate lateral and rotational guidance of the connector 304 during insertion thereof into the port. Thus, the proximal connector portion 344 includes various features that correspond with the features of the various components of the port. For example, the proximal connector portion 344 includes a tapered portion 313 at the proximal end 311 that increases in diameter distally. The tapered portion 313 facilitates pre-insertion and insertion axial (e.g., lateral) guidance of the proximal connector portion 344 through, e.g., the opening 202 of the cover plate 204 for improving ease of insertion of the connector 304.

The proximal connector portion 344 further includes a main cam slot 316 formed axially along an inner surface 317 thereof. The cam slot 316 engages with a port adapter (e.g., port adapter 115) to provide coarse, quasi-helical guided rotation of the proximal connector portion 344 for initial rotational pre-alignment of the connector 304 and thus, the optical fiber 308 angularly aligned and fixed therewith, with the port adapter. The proximal connector portion 344 also includes a circumferential catch 318 that is distal to the tapered portion 313 and includes a conical ramp 319 having an outer diameter decreasing in the distal direction. The conical ramp 319 facilitates proper positioning of the barrel member 340 within the port by providing a declined plane that encourages proximal axial movement of the barrel member 340 into the port.

The ferrule 350 is substantially cylindrical in shape (e.g., a straight tip (ST) ceramic ferrule) and secures a proximal end 357 of optical fiber 308 at the proximal end 311 of the connector 304. The ferrule 350 is comprised of a ceramic material (e.g., zirconia) or metallic material. The ferrule 350 facilitates correct positioning of the proximal end 357 of the optical fiber 308 within the port. A proximal end 341 of the ferrule 350 includes a tapered portion 342 that increases in diameter distally, and a cavity (or air-gap) 330 that decreases in diameter distally. The cavity 330 surrounds the proximal end 357 of the optical fiber 308, in some embodiments, as a counterbore. In some embodiments, the cavity 330 may comprise a concave counterbore that is square-shape, triangle-shaped, a round-shape, etc. In some embodiments, this concave, bowl-like, or otherwise inwardly converging counterbore shape may help to facilitate convergence of beams (e.g., beam 160) into the optical fiber 308. The cavity 330 also helps absorb heat produced by beams received at the proximal end 341 of the ferrule 350, for example, when the connector 304 is used in high-power laser applications.

The ferrule 350 is supported within an interior of the proximal connector portion 344 via a cylindrical ferrule body 315, to which the ferrule 350 is fixedly coupled. As shown in FIGS. 3B-3C, the ferrule 350 is disposed against an inner wall 364 of the ferrule body 315 and extends from a proximal end 339 of the ferrule body 315, which is slidably coupled to the medial portion 346, into the proximal connector portion 344. Accordingly, an inner diameter of the medial portion 346 is tightly toleranced to an outer diameter of ferrule body 315. As an example, the ferrule body 315 is comprised of a plastic material.

As shown in FIG. 3B, an axial spring 332 is circumferentially disposed around the ferrule body 315 between the proximal end 339 of ferrule body 315 and the medial portion 346. The spring 332 is supported between a flange 333 at the proximal end 339 of the ferrule body 315 and a proximal wall 334 of the medial portion 346. The spring 332 provides an axial spring-loaded force on the ferrule body 315 in the proximal direction to facilitate correct positioning of the ferrule 350 when the connector 304 is connected to the port. In other words, the spring 332 is configured to enable spring-loaded axial registration of the ferrule body 315 against the port when the connector 304 is fully inserted into the port. For example, when the connector 304 is inserted into the port, the spring 332 compresses and the ferrule 350 retracts axially.

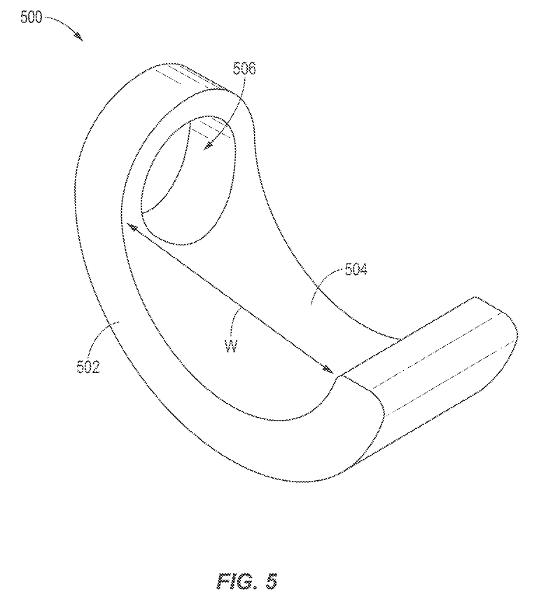

To prevent the ferrule body 315 from sliding out of the proximal connector portion 344 as a result of the axial force from spring 332, a retaining clip 335 is attached on a distal end 337 of the ferrule body 315, which catches against a medial wall 336 within the medial portion 346. To prevent the optical fiber 308 from breaking during attachment of the retaining clip 335, a holder may be inserted through a slot in the housing 320 to hold and support the ferrule body 315 at the distal connector portion 348. An example of the holder is described in further detail with reference to FIG. 5.

As shown in FIGS. 3B-3C, the ferrule body 315 further includes an aperture 352 therethrough near the proximal end 339 of the ferrule body 315. To couple the ferrule body 315 to the ferrule 350, an adhesive (e.g., epoxy, Ultraviolet (UV) cure adhesive, UV-heat hybrid cure adhesive, or other medical grade adhesive, such as EP62, 353ND) may be applied via the aperture 352.

A protective tube 360 is disposed within the ferrule body 315 and against a distal end 366 of the ferrule 350. The protective tube 360 is disposed around the optical fiber 308 and at least partially along a length thereof. The protective tube 360 surrounds a junction point 354 of the optical fiber 308 that is disposed between the ferrule 350 and the ferrule body 315. Thus, the protective tube 360 protects the optical fiber 308 by preventing the optical fiber 308 from breaking or cracking at or near the junction point 354. As an example, the protective tube 360 is a stainless steel hypo tube. An adhesive (e.g., epoxy, UV (ultraviolet) cure adhesive, UV-heat hybrid cure adhesive, or other medical grade adhesive) may be applied to couple the protective tube 360 to the distal end 366 of the ferrule 350. The adhesive between the protective tube 360 and the ferrule 350 withstands high temperatures (e.g., up to 150 °C. (Celsius)) produced by beams propagated onto the optical fiber 308 during ophthalmic procedures (e.g., cataract surgeries, corneal transplants, glaucoma surgeries, LASIK surgeries, refractive lens exchanges, vitreoretinal procedures, trabeculectomies, keratotomy procedures, and/or keratoplasty surgeries, etc.).

The protective tube 360 also helps prevent the optical fiber 308 from breaking while the connector 304 is coupled to the port. Even though the ferrule 350 and a port adapter (e.g., port adapter 115) hold the optical fiber 308 in position, if the connector 304 and/or the cable 310 are moved in any direction other than into/out of the port, the protective tube 360 prevents bending or breaking of the optical fiber 308. In other words, the optical fiber 308 is unlikely to break because the protective tube 360 prevents excessive bending of the optical fiber 308 when the connector 304 and/or cable 310 are moved.

A sleeve 362 is disposed over at least a portion of the protective tube 360 and the optical fiber 308 and within the ferrule body 315. The sleeve 362 extends from the distal end 337 of the ferrule body 315 and into the cable 310. The sleeve 362 is comprised of a material (e.g., polyimide) configured to withstand high temperatures (e.g., up to 150° C.) produced by beams propagated onto the optical fiber 308 during ophthalmic procedures, and to protect the optical fiber 308 from moisture. To couple the sleeve 362 and the ferrule body 315, an adhesive (e.g., epoxy, UV cure adhesive, UV-heat hybrid cure adhesive, or other medical grade adhesive) is applied between the sleeve 362 (e.g., on its outer surface) and the ferrule body 315.

As best seen in FIG. 3C, the optical fiber 308 comprises an uncoated portion (or bare portion) 356 (illustrated by dashed lines) and a coated portion 358. The uncoated portion 356 includes a portion of the optical fiber 308 that has been stripped of its coating, and the coated portion 358 includes a portion of the optical fiber 308 where the coating is still intact. The uncoated portion 356 extends proximally through the ferrule 350 from the junction point 354, and the coated portion 358 extends distally from the junction point 354 and the ferrule 350.

A crystal bond or wax is applied to the uncoated portion 356 at the proximal end 357 of the optical fiber 308 that is partially disposed in the cavity 330 of the ferrule 350. The crystal bond protects the optical fiber 308 from breaking when the optical fiber 308 is polished. After the optical fiber 308 has been polished, the crystal bond is washed away before use of the connector 304. Using the crystal bond at the proximal end 357 of the optical fiber 308 eliminates any requirements for an adhesive at the proximal end 357. By eliminating adhesives at the proximal end 357, the optical fiber 308 is better suited for high-power applications, e.g., relative to optical fibers with adhesives that melted in high-power applications.

By including the ferrule 350 and the optical fiber 308 with the uncoated and coated portions 356 and 358, the connector 304 can be used in high-power laser applications (e.g., involving power outputs greater than 1.5 W) because adhesives, which are often used with glass fibers, can be omitted from the proximal end 357 of the optical fiber 308. In other words, the adhesives that melt in the connectors used for high-power laser applications can be eliminated from the proximal end 357 of the optical fiber 308. As a result, a wide variety of optical fibers, such as a germanium dioxide (GeO2) fiber, a sapphire fiber, a glass fiber, a fluoride fiber, a zirconium barium lanthanum aluminum sodium fluoride (ZBLAN) fiber, a hollow-core fiber, etc., can be used with the connector 304. Thus, the connector 304 can be used in both low-power and high-power laser applications.

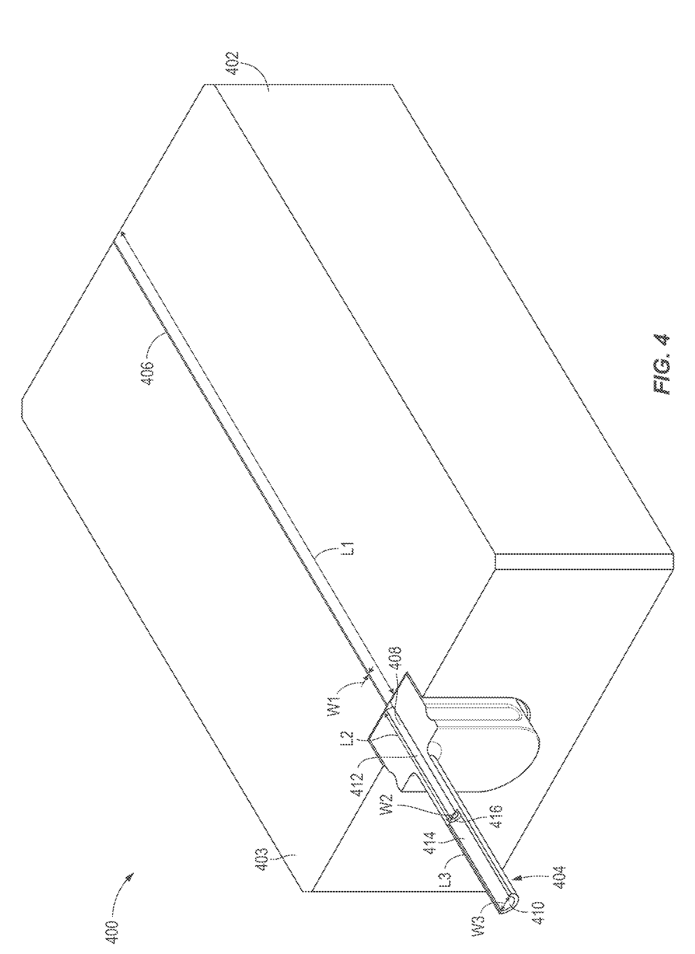

FIG. 4 shows a perspective view of an example connector assembly tool 400, according to certain embodiments. The connector assembly tool 400 can be used to couple an optical fiber with a protective tube and a ferrule, such as the optical fiber 308, the protective tube 360, and the ferrule 350 shown in FIGS. 3A-3C. The connector assembly tool 400 includes a base 402 with a first surface 403 and a semi-circular mount 404 protruding laterally from the base 402.

The base 402 includes a first semi-circular portion 406 configured to hold an optical fiber (e.g., optical fiber 308). The first semi-circular portion 406 has a first width (W1) between 0.001 in (inches) and 0.100 in (e.g., between 0.005 in and 0.950 in, 0.010 in and 0.900 in, or 0.015 in and 0.850 in), and a first length (L1) between 1 in and 10 in (e.g., 2 in and 9 in, 3 in and 8 in, or 4 in and 7 in).

The semi-circular mount 404 includes a second semi-circular portion 408 configured to hold a protective tube (e.g., protective tube 360) and a third semi-circular portion 410 configured to hold a ferrule (e.g., ferrule 350). The second semi-circular portion 408 is disposed proximal to the first semi-circular portion 406, for example, relative to the base 402. The second semi-circular portion 408 includes an inner surface 412 against which the protective tube may be positioned. The second semi-circular portion 408 has a second width (W2) between 0.01 in and 0.90 in (e.g., between 0.02 in and 0.85 in, 0.03 in and 0.80 in, or 0.04 in and 0.75 in), and a second length (L2) between 0.1 in and 1.2 in (e.g., between 0.2 in and 1.1 in, 0.3 in and 1.0 in, or 0.4 in and 0.9 in).

The third semi-circular portion 410 is disposed proximal to the second semi-circular portion 408, for example, relative to the base 402. The third semi-circular portion 410 includes an inner surface 414 and an inner wall 416 against which the ferrule may be positioned. The third semi-circular portion 410 has a third width (W3) between 0.07 in and 1.00 in (e.g., between 0.08 in and 0.90 in, 0.09 in and 0.80 in, or 0.10 in and 0.70 in), and a third length (L3) between 0.1 in and 1.2 in (e.g., between 0.2 in and 1.1 in, 0.3 in and 1.0 in, or 0.4 in and 0.9 in).

As shown in FIG. 4, the first semi-circular portion 406 is longitudinally aligned with the second semi-circular portion 408 and the third semi-circular portion 410. The first semi-circular portion 406, the second semi-circular portion 408, and the third semi-circular portion 410 are aligned to facilitate insertion of the optical fiber into the protective tube and the ferrule. The connector assembly tool 400 is coated with a layer of wax to prevent self-curing during the coupling of the optical fiber, the protective tube, and the ferrule.

The positioning and sizing of the semi-circular portions 406, 408, 410 provides precise alignment of an optical fiber (e.g., optical fiber 308) with a protective tube (e.g., protective tube 360) and a ferrule (e.g., ferrule 350), maintains integrity of the optical fiber, and prevents bending or breaking of the optical fiber upon insertion into the protective tube and the ferrule. Such alignment is particularly useful when inserting a fragile optical fiber (e.g., GeO2 fiber or sapphire fiber) into the protective tube and the ferrule because it ensures proper insertion of the optical fiber into the protective tube and the ferrule. Further, when a connector (e.g., connector 304) is disconnected from a port and the optical fiber is replaced, the connector assembly tool 400 provides consistent alignment between the new optical fiber, protective tube, and ferrule. Accordingly, during the assembly of different connectors, an optical power of the transmitted light remains constant across each assembled connector because the optical fiber is consistently aligned with the protective tube and the ferrule.

FIG. 5 shows a perspective view of an example ferrule body holder tool 500, according to certain embodiments. The ferrule body holder tool 500 can be used to hold a ferrule body within a connector during attachment of a retaining clip to the ferrule body, such as during the attachment of the retaining clip 335 to the ferrule body 315 in the connector 304 shown in FIG. 3B. For example, the ferrule body holder tool 500 is inserted through a slot in a barrel member (e.g., barrel member 340) to hold a distal end (e.g., distal end 337) of the ferrule body (e.g., ferrule body 315), while the retaining clip is attached thereto.

The ferrule body holder tool 500 includes a base 502 with an inner surface 504 and an aperture 506 therethrough. The base 502 and the inner surface 504 have a semi-circular shape that is configured to hold a ferrule body (e.g., ferrule body 315). The inner surface 504 has a width (W) between 0.05 in and 0.50 in (e.g., between 0.10 in and 0.45 in, 0.15 in and 0.40 in, or 0.20 in and 0.35 in). The aperture 506 can be used by a user to hold (e.g., via tweezers or other similar tool) the ferrule body holder tool 500 during the insertion and removal thereof into and out of a connector (e.g., connector 304).

By using the ferrule body holder tool 500 during the attachment of a retaining clip to a ferrule body, the ferrule body holder tool 500 provides stability to, and supports the ferrule body while also preventing breakage of an optical fiber (e.g., optical fiber 308) therein. In other words, the ferrule body holder tool 500 helps hold or maintain the position of the ferrule body, so that it does not move relative to its longitudinal axis while the retaining clip is coupled thereto. Holding the position of the ferrule body thereby avoids accidental bending and breaking of an optical fiber (e.g., optical fiber 308) therein.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A connector for an ophthalmic surgical device comprising:
a housing configured to be gripped by a user for coupling to a surgical console;
an optical fiber disposed within the housing;
a ferrule body disposed around at least a portion of the optical fiber;
a protective tube disposed within the ferrule body; and
a ferrule coupled to the ferrule body and the protective tube, wherein:
the ferrule defines a cavity at a proximal end of the ferrule, and
the optical fiber is at least partially disposed within the cavity.

2. The connector of claim 1, wherein:
a proximal end of the optical fiber comprising a crystal bond, and
the proximal end of the optical fiber is at least partially disposed within the cavity of the ferrule.

3. The connector of claim 1, wherein an uncoated portion of the optical fiber extends proximally from the protective tube and through the ferrule.

4. The connector of claim 1, wherein the protective tube surrounds a junction point of the optical fiber that is disposed between the ferrule and the ferrule body.

5. The connector of claim 1, wherein the protective tube is disposed against a distal end of the ferrule.

6. The connector of claim 1, wherein the cavity at the proximal end of the ferrule has an inwardly converging shape.

7. The connector of claim 1, wherein the cavity at the proximal end of the ferrule surrounds a proximal end of the optical fiber.

8. The connector of claim 1, wherein the ferrule body comprises an aperture through which an adhesive is applied to couple the ferrule body with the ferrule.

9. A connector assembly for an ophthalmic surgical device comprising:
a cable configured to be coupled with the ophthalmic surgical device; and
a connector coupled to a proximal end of the cable, the connector comprising:
a housing configured to be gripped by a user for coupling to a surgical console;
an optical fiber disposed within the housing;
a ferrule body disposed around at least a portion of the optical fiber;
a protective tube disposed within the ferrule body; and
a ferrule coupled to the ferrule body and the protective tube, wherein:
the ferrule defines a cavity at a proximal end of the ferrule, and
the optical fiber is at least partially disposed within the cavity.

10. The connector assembly of claim 9, wherein:
a proximal end of the optical fiber comprising a crystal bond, and
the proximal end is at least partially disposed within the cavity of the ferrule.

11. The connector assembly of claim 9, wherein an uncoated portion of the optical fiber extends proximally from the protective tube and through the ferrule.

12. The connector assembly of claim 9, wherein the protective tube surrounds a junction point of the optical fiber that is disposed between the ferrule and the ferrule body.

13. The connector assembly of claim 9, wherein the cavity at the proximal end of the ferrule has an inwardly converging shape.

14. A connector assembly tool comprising:
a base comprising:
a first semi-circular portion configured to hold an optical fiber; and
a semi-circular mount protruding laterally from the base, the semi-circular mount comprising:
a second semi-circular portion configured to hold a protective tube; and
a third semi-circular portion configured to hold a ferrule.

15. The connector assembly tool of claim 14, wherein the first semi-circular portion, the second semi-circular portion, and the third semi-circular portion are aligned to facilitate insertion of the optical fiber into the protective tube and the ferrule.
